(19) United States
(12) Patent Application Publication
(10) Pub. No.: US 2012/0109319 A1
(43) Pub. Date: May 3, 2012

(54) INTERBODY CAGE

(76) Inventor: Michael Perisic, Zollikon (CH)

(21) Appl. No.: 13/383,483

(22) PCT Filed: Jul. 14, 2010
(86) PCT No.: PCT/IB10/01725

§ 371 Date: Jan. 11, 2012

(30) Foreign Application Priority Data
Jul. 14, 2009 (CH) 01105/09
Dec. 2, 2009 (CH) 01849/09

(51) Int. Cl. A61F 2/44

(52) U.S. Cl. 623/17.16

(57) ABSTRACT

The invention relates to an interbody cage, including a cage body (1) comprising a first and second vertebral bearing element (2a, 2b) each provided with a surface (3a, 3b) capable of bearing against upper and lower vertebrae (4a, 4b), respectively, wherein the first and second vertebral bearing elements (2a, 2b) are capable of being angled relative to one another. The interbody cage further comprises an expansion element (5) to be inserted into the interbody space between the two vertebrae (4a, 4b), then rotatably actuated to expand the interbody space such that the cage body (1) can be inserted therein. The expansion element (5) or other means (6) are arranged so as to vary, in situ, the angle of the first vertebral bearing element (2a) relative to the second vertebral bearing element (2b) of the cage body (1) when the cage body is inserted into the interbody space.

The present invention relates to the medical field, and more particularly to an interbody cage.

Certain pathologies of the human spine, such as degenerated discs and facettes diseases, and dislocation of vertebrae, compromise the support capacity of the column and the sharing of the load.

The treatment of such pathologies in their advanced stages is achieved by various stabilization systems with intra-discal implants such as interbody cages, whether or not coupled with extra-discal systems, which combine the use of vertebral screws and plates or rods. Such intra-discal implants have significantly improved the treatment of pathologies of the human spine, in restoring the intervertebral space, which results in the decompression of the nerve roots and the acceleration of bony fusion of the adjacent vertebrae together.

Impactation cages represent an important category among interbody cages. These cages, which have a substantially parallelipipedic shape, are inserted between the vertebrae by impactation. The downside of these cages is the difficulty of their insertion into the intervertebral space, notably through posterior or unilateral approaches notably transforaminal, lateral or anterio lateral. The dents which are integrated on the superior and inferior surfaces of the cage for the purpose of anchoring the cage into the vertebral plates to prevent its expulsion, once it is in its final position, represent an additional obstacle to its insertion.

US200810269758 discloses an interbody cage comprising two elements, the first, in a “U” shape, serves as means of insertion, of dilatation (by 90° rotation) and of permanent distraction, and the other as support body for the first element. However, that invention makes of the “U” shape element the main stabilization element of the cage, for the intervertebral segment, i.e. the two instrumented vertebrae mainly engage with that first element, while the second element only serves as buttress supporting the first element.

The purpose of the present invention is to provide an interbody cage which may be introduced into the interbody space without need for impactation, said cage also providing means to reproduce an angle between the two vertebrae which corresponds or is close to the natural angle of lordosis of the instrumented segment.

According to the invention, this goal is achieved by an interbody cage comprising a cage body which comprises a first and second vertebral bearing element, each having one surface designed to bear respectively against a superior and inferior vertebra, and where the first and second vertebral bearing elements are capable of being angled one relative to one another. The interbody cage also comprises an expansion element the purpose of which is to be introduced between the two vertebrae and thereafter rotated so as to expand the interbody space in order to introduce the body of the cage. The expansion element or other means are arranged in order to vary in situ the angle of the first vertebral bearing element relative to the second vertebral bearing element of the cage body once the cage body is introduced into the interbody space.

The characteristics of the invention will appear more clearly from the description of various embodiments, which are solely provided as examples and are not limitative, and in which references will notably be made to the horizontal plane of the cage which will be assumed to be in a parallel plane to the axial or transversal plane of the human body, this horizontal plane corresponding to the position of the cage in its length when it is in its final position between two vertebrae. In addition, the anterior side of the cage body means the side that is adjusted against the interbody space just before the introduction of said body in that space, and the posterior side of the cage body means the side opposite to the anterior side. The description of these various embodiments refers to the attached schematic Figures in which:

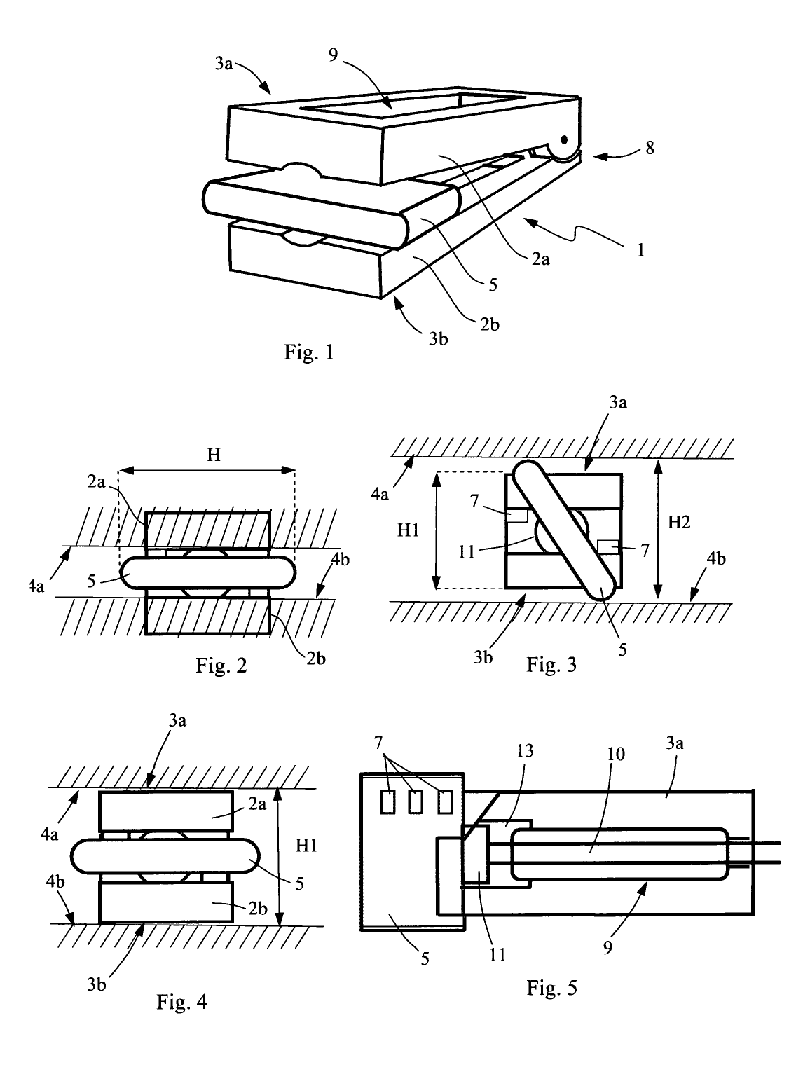

FIG. 1 represents a perspective view of the interbody cage according to a first embodiment;

FIG. 2 represents a view of the anterior side of the cage of FIG. 1, when the expansion element is introduced into the interbody space between two vertebrae;

FIG. 3 represents a view of the anterior side of the cage of FIG. 1, when the expansion element is maneuvered in rotation in order to expand the interbody space;

FIG. 4 represents a view of the anterior side of the cage of FIG. 1, when the cage body is introduced into the interbody space;

FIG. 5 represents a view from the top of the cage of FIG. 5;

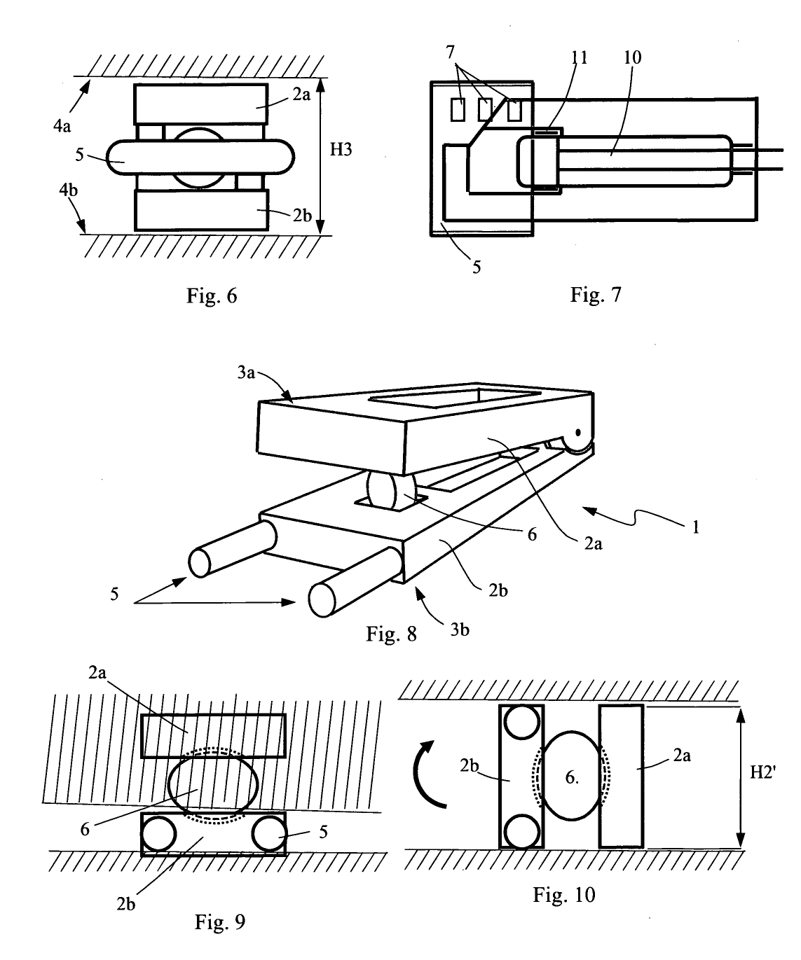

FIG. 6 represents a view of the anterior side of the cage of FIG. 1 in the interbody space when the expansion element is partially retracted into the cage body;

FIG. 7 represents a view from the top of the cage of FIG. 6;

FIG. 8 represents a perspective view of an interbody cage according to a second embodiment;

FIG. 9 represents a view of the anterior side of the cage of FIG. 8, when the expansion element is introduced into the interbody space between two vertebrae;

FIG. 10 represents a view of the anterior side of the cage of FIG. 8 after rotation of the cage by 90° to distract the interbody space;

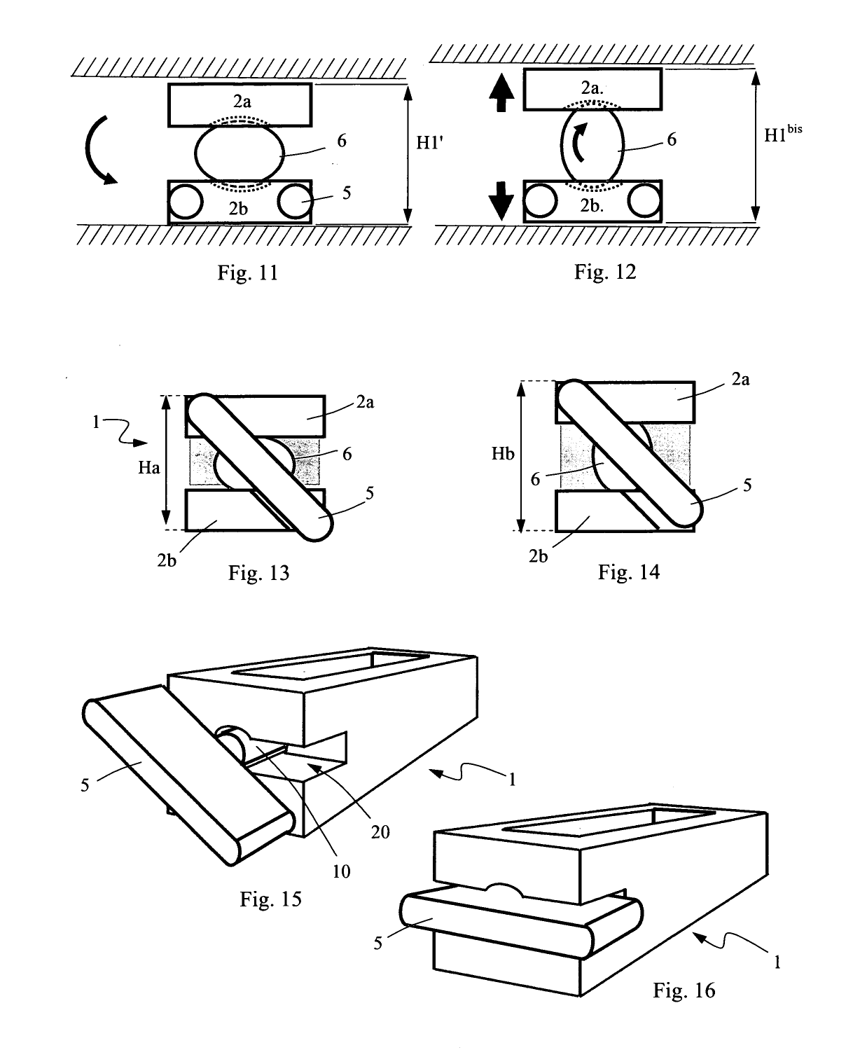

FIG. 11 represents a view of the anterior side of the cage of FIG. 8 in the interbody space after counter-rotation of the cage of 90°;

FIG. 12 represents a view of the anterior side of the cage of FIG. 8 in the interbody space after expansion of the cage;

FIG. 13 represents a view from the top of an interbody cage according to a third embodiment;

FIG. 14 represents a similar view to FIG. 13 after expansion of the cage;

FIGS. 15 and 16 represent a first and, respectively, a second perspective view of an interbody cage according to a fourth embodiment.

According to the first embodiment represented in FIGS. 1 to 7, expansion element 5 is integrated in an expandable cage for posterior or posterior-lateral approaches. This cage comprises a first and second vertebral bearing element 2a, 2b which define the body 1 of the cage, which has an elongated shape. Each vertebral bearing element 2a, 2b has one surface 3a, 3b, which may bear against a superior 4a and respectively an inferior vertebra 4b. The two vertebral bearing elements 2a, 2b are articulated one relative to one another at the posterior level of the cage 1 by means of a hinge 8, so that each vertebral bearing element 2a, 2b may be angled to reproduce the natural angle of lordosis of the instrumented segment. Each vertebral bearing element 2a, 2b preferably contains a cavity 9 which may contain bone graft. The means of expansion of the interbody space in which the cage is destined to be introduced contains, according to this first embodiment an expansion element 5 having a substantially rectangular cross-section. This expansion element 5 is located on the anterior side of the body 1 of the cage and is connected to a rod 10 which crosses the body 1 in its longitudinal axis. Rod 10 also crosses a cylindrical base 11 arranged between the two vertebral bearing elements 2a, 2b of the body 1 of the cage, near the anterior side, the superior and inferior sides of the base 11 being arranged in a channel 13 present in each bearing element 2a, 2b.

According to FIG. 2, the expansion element 5 is first introduced into the interbody space. This expansion element 5 is then actuated in rotation in an oblique plane in angle preferably between 60° and 85° relative to the horizontal plane of the cage body, prompting an angular movement to rod 10, by a delivery instrument. This has the effect of expanding the interbody space to a height H2 superior to height H1 of the body 1 of the cage (FIG. 3). At this time, the cage body is pushed in order to be introduced into the interbody space. According to FIG. 4, the expansion element is then rotated back into a horizontal position corresponding to the axial plane of the human body. In this position, the superior and inferior vertebrae bear against the first and second vertebral bearing elements 2a, 2b, respectively. Expansion element 5 may thereafter be easily retracted, at least partially, inside the cage body 1 along its longitudinal axis. While retracting, this expansion element 5 engages with the first and second vertebral bearing elements 2a, 2b and thus exercises a distraction force on said bearing elements 2a, 2b, so as to modify the relative angulation between them. The anterior part of the body of the cage rises and moves from height H1 to height H3 (FIG. 6).

The superior and inferior surfaces of the expansion element 5 comprise three crevasses 7 located along one of its lateral sides so as to engage with the anchoring elements in the shape of notches 12 located on the first and second vertebral bearing elements 2a, 2b in order to lock expansion element 5 in various axial positions along de longitudinal axis of body 1. Each axial position of expansion element 5 thus corresponds to a predetermined angulation of the first bearing element 2a relative to the second bearing element 2b of body 1 of the cage. A variation (not represented) consists in multiplying the notches, or to locate the notches on the expansion element and the crevasses on the vertebral bearing elements 2a, 2b. The expansion of the two bearing elements 2a, 2b of the body of the cage may thus be modulated by the level of retraction of the expansion element 5, and the stabilization in this position by the choice of the notch 7 which is the most appropriate for the desired angle. After completion of the angulation of the two bearing elements 2a, 2b in their final angle, a rotation of rod 10 (in the direction contrary to the direction used to expand the interbody space) enables to separate the rod from the base 11 so that it may be extracted from the implant.

In a variation (not represented), the vertebral bearing elements of the cage do not engage with the superior and inferior surfaces of the expansion element, but with the cylindrical base. In that variation, it is the axial movement of the base along the channel which provokes the angulation of the first and second vertebral bearing elements. It is also the base which constitutes the final bearing point of the vertebral bearing elements. That base may have a thread around its surface and the surfaces of the channel in each bearing element may contain a portion of a corresponding thread, so that the base may move axially by rotation of the rod until the desired position, whence it may be blocked without being screwed, for instance through a system of notched wheels.

According to the second embodiment of the invention as represented in FIGS. 9 to 12, the expansion element 5 is constituted by two rods which are fixed on the anterior side of the body 1 of the cage on one of the bearing elements 2a, 2b in the continuation of the longitudinal sides of body 1. In lieu of the two rods, the expansion element may be in another protruding shape.

In this embodiment of the invention, the two rods are introduced in the interbody space, and then, in contrast to the first embodiment, it is not the distraction element but the body of the cage which is actuated in 90° rotation to expand the interbody space of a height H2′ corresponding to the width of the cage (FIG. 10). The cage is then pushed in this position until its final desired location within the interbody space, at which stage a counter-rotation is exercised on the cage (FIG. 11), so as to bear the vertebral bearing elements 2a, 2b against the respective vertebrae, which are then separated of a distance H1′, corresponding to the height of the cage before its expansion.

An oval shaped wheel 6 is arranged within the body 1, on which the two vertebral bearing elements 2a, 2b lean. The wheel 6 is connected in its center to a rod (not represented) which crosses the cage body in its longitudinal axis and emerging from the posterior face of said body. According to FIG. 12, this wheel 6 may thus be activated in rotation by actuating an angular movement to this rod to vary the angulation between the first and the second bearing elements 2a, 2b, once the body of the cage is introduced in the interbody space, such that height H1^{bis} at the level of the anterior part of the cage body is higher than height H1′ of the cage in its non-expanded position. This enables to reproduce the natural lordotic angle of the instrumented segment (in the case of a cage for posterior or posterior-lateral approaches). The wheel may be blocked in a predefined position by any technically appropriate mean.

According to a third embodiment of the invention, as represented in FIGS. 13 and 14, the interbody cage comprises, alike to the second embodiment, a cage body comprising a first and second vertebral bearing element 2a, 2b, which are each in contact with an oval wheel 6 arranged between the two elements 2a and 2b, which wheel may be actuated in rotation around the longitudinal axis of the cage body. In contrast to the second embodiment, this cage comprises an expansion element 5 which arranged in an oblique plane relative to the horizontal plane of the cage, on the anterior side of the cage body. According to this embodiment, the cage may be rotated with an angle inferior to 90°, and preferably between 60 and 85° (depending on the ratio between the height and the width of the cage). The expansion element 5 is rigidly connected to at least one of the vertebral bearing elements 2a, 2b on the anterior side of the body 1 of the cage, such that one of the lateral sides of said expansion element 5 is in the prolongation of one of the longitudinal sides of said body. Expansion element 5 is laid out in an oblique plane relative to the horizontal plane of the cage, the other one of the lateral sides of said expansion element 5 being at the level of the other one of the vertebral bearing elements 2a, 2b in the prolongation of the other longitudinal sides of the body. However, expansion element 5 is not rigidly connected to such other bearing element so that the cage body may be expanded by rotation of wheel 6 which has preferably in an oval shape. The anterior side of the cage body moves thus from a height Ha before expansion to a height Hb after expansion.

According to a fourth embodiment of the invention as illustrated in FIGS. 15 and 16, the interbody cage comprises one body 1, preferably a mono-block, and one expansion element 5 arranged on the anterior side of body 1 of the cage and connected in its median part to a rod 10 which crosses body 1 along its longitudinal axis. Expansion element 5 is arranged to be activated in translation and in rotation. Expansion element 5 is actuated in translation in order to be introduced into the interbody space between two vertebrae, and is thereafter actuated in rotation to expand the interbody space in order to be able to introduce the body 1 of the cage therein; the expansion element is then rotated back into the horizontal plane of the cage and then retracted into a housing 20 located on the anterior side of the body 1 of the cage once the latter is in its final position between the two vertebrae, so as to secure the expansion element and limit its protruding beyond the anterior side of the cage. In a non-illustrated variation, the depth of the housing is more important, and preferably spans over more than half of the body, thus defining a first and second vertebral bearing element. The posterior part of the body has elastic properties in order to vary in situ the angulation of the first vertebral bearing element relative to the second vertebral bearing element according to the principle of the first embodiment, i.e. by retraction of the expansion element between the first and the second along the longitudinal axis of the cage body.

A variation (not represented) consists in inserting in a first step only the expansion element into the interbody space, and thereafter to exercise a rotation between 70° and 90° so as to allow the expansion element to distract said interbody space, and then to push the expansion element (without the body of the cage) in that position, which is near perpendicular to the vertebral plates, to the anterior part of the interbody space (in the case of a cage for posterior and postero-lateral approaches). The body of the cage is then introduced by sliding it along the rod, in its upside position, until the front end of the body touches the back end of the expansion element. At that stage, a counter-rotation is exercised on the rod, and the lateral sides of expansion element lose their bearing against the vertebral plates, which then lean directly on the superior and inferior surfaces of the cage body.

The vertebral bearing elements, the expansion element and the insertion rods may be made of different materials, including in softer materials in order to give the cage a shock-absorbing property.

The first and second vertebral bearing elements 2a, 2b according any of the first three embodiments, may be connected one to another by another means than a hinge, so as to allow an increase of the height of the cage body to the level of, or close to, its posterior portion.

In addition, the expansion element may be replaces by two protruding parts positioned beyond and in the continuation of the longitudinal sides of the cage body, when they are arranged in the horizontal plane of the cage body, said protruding parts being arranged to be actuated in rotation around the longitudinal axis of the body in order to obtain the same expansion effect on the interbody space.

All variations may also apply to cages which are not destined to be introduced by a posterior approach, inter alia, cages for trans-foraminal, lateral and antero-lateral approaches. In those cases (not represented), the hinge will always be arranged in the posterior part of the cage when it is in its final position (but along one of its flanks, if the cage is considered in its longest dimension), but the retracting of the flap provokes a lateral opening of the cage if the cage is considered in its longest dimension.

1. Interbody cage comprising a cage body (1) which comprises a first and a second vertebral bearing element (2a, 2b) each having one surface (3a, 3b) to bear against respectively a superior and inferior vertebra (4a, 4b), wherein the first and second bearing element (2a, 2b) are capable of being angled one relative to the other, and wherein the cage also comprises an expansion element 5 destined to be introduced in the interbody space between two vertebrae (4a, 4b) and then actuated in rotation to expand the interbody space in order to be able to introduce the cage body (1), the expansion element (5) or other means (6) being arranged to vary in situ the angulation of the first vertebral bearing element (2a) relative to the second vertebral bearing element (2b) of the cage body (1) when the cage body is introduced in the interbody space.

2. Interbody cage of claim 1, wherein the cage body (1) has an elongated shape, and in which the expansion element (5) is arranged on one of the lateral sides or on one of the longitudinal sides of the cage body (1).

3. Interbody cage of claim 2, wherein the expansion element (5) is also arranged, or may be arranged by rotation, in an angulated plane relative to the horizontal plane of the cage body (1).

4. Interbody cage of claim 3, wherein said angulated plane is in an angle between 40° and 85° with the horizontal plane of the cage body (1).

5. Interbody cage of claim 3 or 4, wherein the expansion element (5) is also arranged to be actuated in translation along the longitudinal axis of the cage body (1) or along a perpendicular axis to this longitudinal axis in order to be introduced, at least partly, within said body (1).

6. Interbody cage of claim 5, wherein the first and second vertebral bearing element (2a, 2b) of the cage body (1) are arranged to engage with the expansion element (5) in order that the angulation of the first vertebral bearing element (2) relative to the second vertebral bearing element (2b) depends on the axial position of the expansion element (5) along the longitudinal axis of the cage body (1) or along the perpendicular axis to said longitudinal axis.

7. Interbody cage of claim 6 comprising an indexation system to lock the expansion element (5) into different axial positions corresponding to different angulations of the first vertebral bearing element (2a) relative to the second vertebral bearing element (2b) of the cage body (1).

8. Interbody cage of claim 1, 2, 3 or 4, wherein the means arranged to angulate in situ the first and the second vertebral bearing element (2a, 2b) of the cage body (1) one relative to the other contain a cam (6) arranged to be actuated in rotation, the profile of the cam (6) allowing to vary the angulation of the first vertebral bearing element (2a) relative to the second vertebral bearing element (2b) as a result of the angular position of said cam (6).

9. Interbody cage of claim 1, wherein the first and the second vertebral bearing elements (2a, 2b) of the cage body (1) are angled together by a hinge (8).
